(19) United States
(12) Patent Application Publication
(10) Pub. No.: US 2024/0416059 A1
(43) Pub. Date: Dec. 19, 2024

(54) VARIANCE ANALYSIS, PREDICTION, AND DISPLAY FOR RESPIRATORY - RELEVANT CURVES AND LOOPS

(71) Applicant: KONINKLIJKE PHILIPS N.V., EINDHOVEN (NL)

(72) Inventors: Rafael Wiemker, Hamburg (DE); Roberto Buizza, Eindhoven (NL); Jaap Roger Haartsen, Eindhoven (NL); Cornelis Petrus Hendriks, Eindhoven (NL); Thomas Koehler, Hamburg (NL); Michael Polkey, London (GB); Joerg Sabczynski, Hamburg (DE); Nataly Wieberneit, Hamburg (DE)

(21) Appl. No.: 18/741,855

(22) Filed: Jun. 13, 2024

Related U.S. Application Data
(60) Provisional application No. 63/472,845, filed on Jun. 14, 2023.

(51) Int. Cl. A61M 16/00; A61M 16/04; G16H 20/40

(52) U.S. Cl. CPC A61M 16/024; A61M 16/0051; A61M 16/0066; G16H 20/40; A61M 16/04; A61M 2205/10; A61M 2205/18; A61M 2205/502

(57) ABSTRACT

A mechanical ventilation device includes at least one electronic controller configured to receive two or more respiratory data streams, each spanning multiple breath cycles; generate a confidence band over a breath cycle for each data stream based on statistics of the data stream determined from the multiple breaths spanned by the data stream; and on a display, plot the confidence bands for the data streams on a common graph.

### CROSS-REFERENCE TO RELATED APPLICATIONS

This patent application claims the priority benefit under 35 U.S.C. § 119 (c) of U.S. Provisional Application No. 63/472,845, filed on Jun. 14, 2023, the contents of which are herein incorporated by reference.

The following relates generally to the respiratory therapy arts, mechanical ventilation arts, mechanical ventilation weaning arts, ventilator induced lung injury (VILI) arts, and related arts.

### BACKGROUND

Typical and well-known curves for several quantities which are relevant in mechanical ventilation, e.g., airway pressure, flow, volume, respired gas saturation, thoracic diaphragm excursion, thoracic diaphragm thickening, etc., are plotted against time (‘curves’) or against each other (‘loops’). These curves and loops are known to give valuable cues on the diagnosis, state, progression, prognosis, and therapy response of mechanically ventilated patients.

Clinicians can use pressure-volume (P-V) loops and parameter estimations of the respiratory system (e.g., resistance and compliance) to monitor the patient status and to select safe ventilation therapy settings for the patient. Most accurate results are obtained using the transpulmonary pressure, which is accessible with esophageal catheter measurements. However, often a catheter is not present, in which case the above estimations are impacted by the presence of a patient effort and the chest wall mechanics. Modern ventilators, therefore, avoid a patient effort and ignore chest wall mechanics when determining the transpulmonary pressure.

Respiratory therapists can use a P-V loop as guidance for choosing the right ventilator settings to avoid atelectasis and overdistension which can lead to ventilation induced lung injury (VILI). Ventilators can display P-V loops on their screen. Ventilators should use the transpulmonary pressure to construct the P-V loop accurately, since the transpulmonary pressure is responsible for the deformation of lung tissue, (i.e., the parenchyma tissue with alveoli). The transpulmonary pressure Pₗ is the difference between a pleural pressure and an alveolar pressure.

When a change in therapy is administered, such as proning of the patient (turning from supine to prone lying body-positioning), changing medication, or changing pressure, volume, targets or other settings on the mechanical ventilator (MV), then the various curves/loops are observed for possible changes in order to assess the effectiveness of the therapeutic change. Since these changes may be subtle or only visible in parts of the curves/loops, a routine question is whether these changes are significant, or mere random fluctuations.

When no therapeutic change is intentionally administered, changes in curves/loops may indicate a certain disease progression. The same question arises whether the changes are significant, or mere random fluctuations.

With both above types of changes, the clinical question arises, whether the magnitude of a change is as expected, or significantly different.

The following discloses certain improvements to overcome these problems and others.

### SUMMARY

In one aspect, a mechanical ventilation device includes at least one electronic controller configured to receive two or more respiratory data streams, each spanning multiple breath cycles; generate a confidence band over a breath cycle for each data stream based on statistics of the data stream determined from the multiple breaths spanned by the data stream; and on a display, plot the confidence bands for the data streams on a common graph.

In another aspect, a mechanical ventilation monitoring or assessment method includes, with at least one electronic controller, receiving two or more respiratory data streams, each spanning multiple breath cycles; generating a confidence band over a breath cycle for each data stream based on statistics of the data stream determined from the multiple breaths spanned by the data stream; and on a display, plotting the confidence bands for the data streams on a common graph.

One advantage resides in providing an improved graphical user interface that facilitates determining whether a change in therapy administered to a patient causes significant changes in curves or are random fluctuations.

Another advantage resides in providing a mechanical ventilator (MV) device with an improved graphical user interface that facilitates determining whether a change in MV settings administered to a patient causes significant changes in curves or are random fluctuations.

Another advantage resides in providing an improved graphical user interface that facilitates determining whether a change in therapy administered to a patient causes an expected change in curves.

Another advantage resides in providing an improved graphical user interface that displays curves with confidence intervals and/or error margins.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation device in accordance with the present disclosure.

FIG. 2 shows an example flow chart of operations suitably performed by the device of FIG. 1.

FIG. 3 shows an example flow chart of the confidence band generation step of the flow chart of FIG. 2.

FIGS. 4-6 shows example curves generated by the device of FIG. 1.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

The disclosed system provides an improved graphical user interface that replaces the usual curve or loop plot with a curve band or loop band plot, in which the width of the curve band or loop band provides a visual quantification of the confidence of the measurement. In general, a wider band corresponds to lower confidence (i.e. higher uncertainty) in the measurement; whereas a narrower band corresponds to higher confidence (i.e. lower uncertainty) in the measurement. The disclosed system leverages the cyclic nature of respiratory data streams such as airway pressure or flow, which repeat with each breath cycle, to provide statistical information from which to extract the band width.

The disclosed system thus provides for automatic computation and offering of a confidence interval for all points of a curve/loop, displayed either graphically as envelopes (i.e., “line thickness”) around the plot line, graphical markers, or as quantifying numbers. The disclosed system also provides for displaying two curves (i.e., before/after, patient/cohort, and so forth) shown synoptically with their respective point-wise confidence intervals, indicating graphically by their varying overlap which parts of the curve/loop are significantly different (as opposed to differences which could be explained by meaningless fluctuations, or measurement scatter). Because the curve or loop band graphically represents the measurement confidence, the user can easily see if a difference between the two curves is likely to be significant: If the bands corresponding to the before/after measured curve or loop overlap then the difference is unlikely to be clinically significant as it is within the measurement error; whereas, if the bands do not overlap then the difference is likely to be clinically significant.

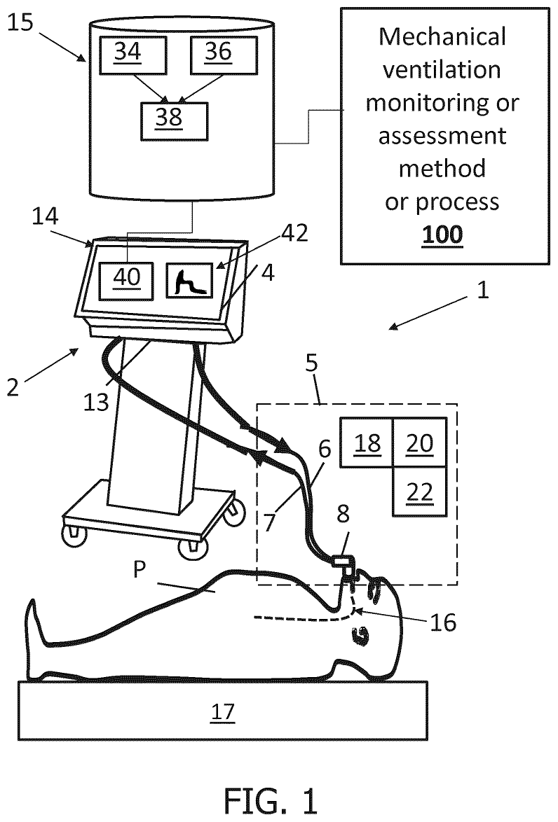

With reference to FIG. 1, a mechanical ventilation device 1 includes a mechanical ventilator 2 for providing ventilation therapy to an associated patient P is shown. As shown in FIG. 1, the mechanical ventilator 2 includes an outlet 4 connectable with a patient breathing circuit 5 to deliver mechanical ventilation to the patient P. The patient breathing circuit 5 includes typical components for a mechanical ventilator, such as an inlet line 6, an optional outlet line 7 (this may be omitted if the ventilator employs a single-limb patient circuit), and a connector or port 8 for connecting with an endotracheal tube (ETT) 16. One or more breathing or respiratory sensors, such as an airway flow meter 18, an airway pressure sensor 20, end-tidal carbon dioxide (etCO₂) sensor 22 (also sometimes referred to as a capnograph), and/or so forth are diagrammatically indicated. The illustrative respiratory sensors 18, 20, 22 are included in the patient breathing circuit 5; however, in other embodiments some or all of the respiratory sensors may be integrated with the mechanical ventilator 2, based on continuity of the breathing circuit 5 (e.g., the pressure delivered to the inlet line 6 may be considered equal to the airway pressure, assuming negligible leakage). The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes an electronic controller 13 (e.g., an electronic processor or a microprocessor), a display device 14, and a non-transitory computer readable medium 15 storing instructions executable by the electronic controller 13. During the mechanical ventilation, the various respiratory sensors 18, 20, 22 monitor parameters of the mechanical ventilation therapy. For example: the illustrative flow meter 18 may provide airflow versus time data; the illustrative airway pressure sensor 20 may provide airway pressure versus time data; and the illustrative etCO₂ sensor 22 may provide end-tidal CO₂ fraction data and may also provide continuous (i.e. sampled at regular intervals) CO₂ versus time data, e.g. a capnograph waveform. It will be appreciated that these waveforms (e.g., air flow, airway pressure, capnograph, et cetera) are at least approximately cyclic as they will cycle with each breath of the patient P. Each breath may be entirely driven by the mechanical ventilator 2 in the case of a fully passive patient; or may include driving contributions from both the mechanical ventilator 2 and breathing effort by the patient P in the case of a patient who is exerting work of breathing (WoB) effort. While the respiratory waveforms measured by the various respiratory sensors 18, 20, 22 are expected to be at least approximately cyclic with the breathing cycle, there may be variations from one breath to the next due to various factors such as variable WoB exerted by the patient, differences in breath length due to breath-by-breath variation in inspiration trigger times (such triggering variability can occur for some mechanical ventilation modes), measurement errors by the respiratory sensors 18, 20, 22, and/or so forth.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth. FIG. 1 illustrates the patient P positioned in the supine position on a diagrammatically indicated patient bed or other support 17. In the supine position, the patient P is lying face-up (unless the head is tilted), that is, with the patient lying on his or her back. The illustrative supine position is typical for a mechanically ventilated patient, since it provides clinicians with access to the patient's mouth and/or to the external portion of the ETT 16, and avoids the potential for the ETT 16 or other parts of the patient breathing circuit from contacting the bed 17. However, the patient orientation may be different from the supine position shown in FIG. 1. For example, in a clinical intervention known as proning, the patient is flipped 180° from the supine position to a prone, i.e. face-down, position which in some cases can improve the efficacy of the mechanical ventilation therapy.

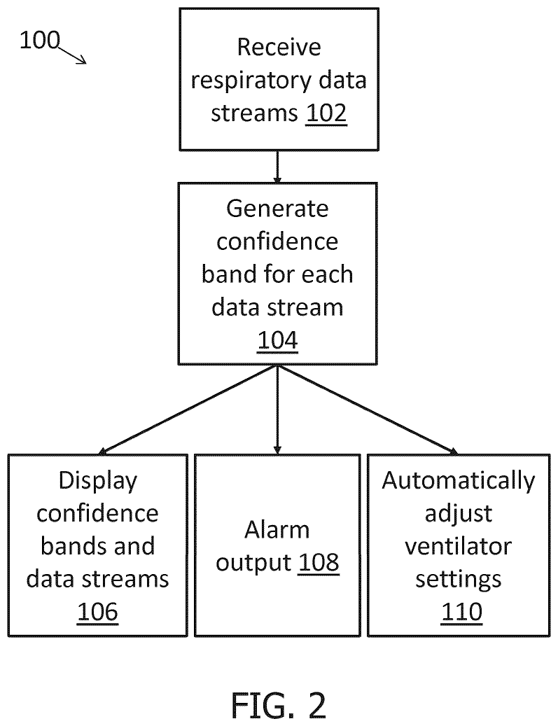

The non-transitory computer readable medium 15 can store instructions executable by the electronic controller 13 to perform a mechanical ventilation monitoring or assessment method or process 100 for monitoring the patient P during mechanical ventilation therapy using the mechanical ventilator 2. With reference to FIG. 2, and with continuing reference to FIG. 1, an illustrative embodiment of a mechanical ventilation monitoring or assessment method 100 is diagrammatically shown as a flowchart. To begin the method 100, the patient P is intubated with the ETT 16 so that mechanical ventilation therapy with the mechanical ventilator 2 can begin.

At an operation 102, two or more respiratory data streams 34, 36 are received by the electronic controller 13. Each respiratory data stream 34, 36 can span multiple breath cycles by the patient P.

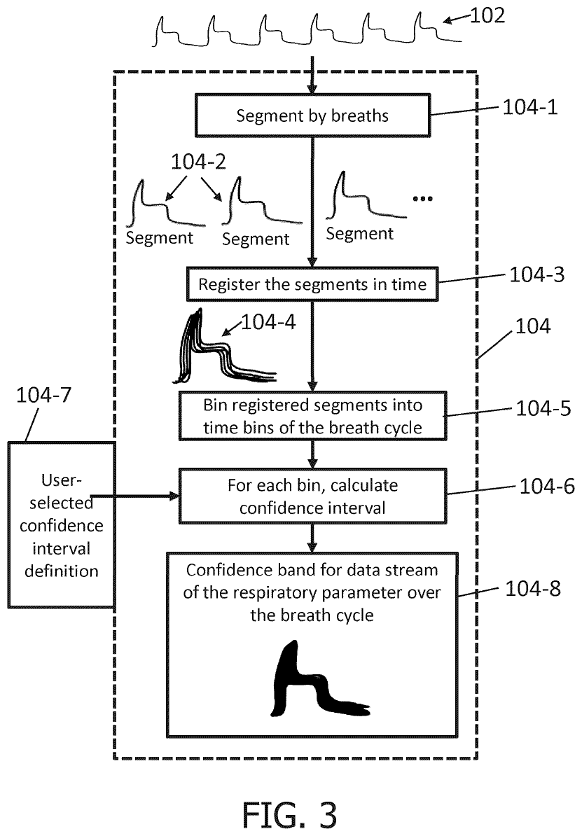

With continuing reference to FIG. 2 and further reference to FIG. 3, at an operation 104, a confidence band 38 can be generated over a breath cycle for each data stream 34, 36 based on statistics of the data stream determined from the multiple breaths spanned by the data stream 34, 36. For example, each respiratory data stream 34, 36 comprises a time sequence of measurements of a respiratory parameter. In the example of FIG. 3, generation of the confidence band 28 over the breath cycle for each data stream 34, 36 includes a step 104-1 of partitioning the data stream 34 or 36 into segments 104-2 each corresponding to a breath. This can be done in various ways, such as in the case of an air flow waveform detecting an abrupt increase in flow into the lungs to indicate the start of the inspiration phase of each cycle. In another approach, a maximum peak (or valley) of the signal in each breath is detected as the segmentation point. These segments are registered in time in an operation 104-3 to produce registered segments 104. The registration 104-3 can employ any suitable one-dimensional rigid or elastic registration approach. For example, an automated registration algorithm can be implemented to sweep over a range of possible registration approaches and parameters, and select the choice which minimizes a sum of the absolute or squared deviations of the segments over the full respiratory cycle. The automated registration algorithm can be used to minimize the area of the confidence band (i.e., to achieve the “sharpest” curve/loop).

The measurements of the registered segments 104-4 are binned in an operation 104-5 into time bins of the breath cycle. The time bins are selected to be narrow enough in time width to provide a desired temporal resolution for the resulting band representation, while being wide enough to provide a statistically significant number of samples for each bin. (The number of samples per bin can also be controlled by including more segments). For each time bin of the breath cycle, in an operation 104-6, a confidence interval of the binned measurements is computed. The confidence band comprises a time sequence of the confidence intervals computed for the time bins. The result is the confidence band 104-8 for the data stream of the respiratory parameter over the breath cycle.

For example, the computing of the confidence interval of the binned measurements includes computing an average or median value of the binned measurements (or another representative value, such as a mode-value). Optionally, any outlier measurements in the bin can be removed prior to computing the confidence band. A statistical spread of the binned measurements is computed. The confidence interval comprises the statistical spread around the average or median value. The statistical spread can be, for example, a standard deviation, a variance, a quartile, or a percentage-quantile.

Since the choice of confidence interval definition (e.g., standard deviation, quantile, an inter-quartile-range (IQR), et cetera) can impact the width of the band, in some embodiments the confidence interval definition can be a user input 104-7 to the confidence interval calculation 104-6, as diagrammatically shown in FIG. 3. For example, as shown in FIG. 1 a user dialog 40 can be presented on the display device 14 of the mechanical ventilator 2 to receive the input 104-7. An updated configuration for a confidence interval metric is received by the user dialog 40 and can be used to compute the confidence interval. In response to receiving an updated configuration for the confidence interval metric, the computing of the confidence intervals is repeated whereby the confidence band is updated in accordance with the updated configuration.

With continuing reference to FIG. 2 and reference back to FIG. 1, at an operation 106, the confidence bands 38 for the data streams 34, 36 are plotted on a common graph 42 and displayed on the display device 14, as diagrammatically shown in FIG. 1.

The mechanical ventilation monitoring or assessment method 100 can be performed for a variety of data streams. In a first embodiment, the received two or more respiratory data streams 34, 36 include a first respiratory data stream 34 for a respiratory parameter acquired of the patient P receiving mechanical ventilation therapy prior to a modification to the mechanical ventilation therapy, and a second respiratory data stream 36 for the respiratory parameter acquired of the patient P receiving the mechanical ventilation therapy after the modification to the mechanical ventilation therapy. In some examples, the modification to the mechanical ventilation therapy comprises proning the patient by moving the patient from a supine position to a prone position on the bed 17, adjusting one or more settings of the mechanical ventilator 2, and so forth. In this embodiment, the operation 104 includes determining whether the modification to the mechanical ventilation therapy has produced a statistically significant change in the respiratory parameter acquired of the patient P based on detection of a non-overlapping portion of the confidence bands 38 generated for the first and second data streams 34, 36. An indication (e.g., on the common graph 42) of whether the modification to the mechanical ventilation therapy has produced a statistically significant change in the respiratory parameter acquired of the patient based on the automatic determination is output on the display device 14.

In another embodiment, the received two or more respiratory data streams 34, 36 include the first respiratory data stream 34 for a respiratory parameter acquired of the patient P over a first time interval, and a second respiratory data stream for the respiratory parameter acquired of the patient P over a second time interval that is after the first time interval and that is not overlapping the first time interval. In this embodiment, the operation 104 includes automatically determine whether there is a change in a condition of the patient P based on whether there is a non-overlapping portion of the confidence bands 38 generated for the first and second data streams 34, 36. When a change in the curve is automatically determined, either an alarm indicative of the change in the curve is output (at an operation 108), and/or a recommended adjustment to one or more parameters of the mechanical ventilation therapy delivered to the patient P is output on the display device 14 (at an operation 110).

In another embodiment, the received two or more respiratory data streams 34, 36 include a first respiratory data stream 34 for a respiratory parameter acquired of a first patient P receiving mechanical ventilation therapy, and a second respiratory data stream 36 for the respiratory parameter acquired of a second patient receiving the mechanical ventilation therapy in which the second patient is different from the first patient.

In another embodiment, the received two or more respiratory data streams 34, 36 include a first set of two or more respiratory data streams 34 for a corresponding two or more respiratory parameters, and a second set of two or more respiratory data streams 36 for the corresponding two or more respiratory parameters. In this embodiment, the operation 104 includes, for the first set of two or more respiratory data streams 34, determining the confidence band 38 based on a covariance between the two or more respiratory data streams 34, 36 of the first set of two or more respiratory data streams 34. For the second set of two or more respiratory data streams 36, determining the confidence band 38 based on the covariance between the two or more respiratory data streams 34, 36 of the second set of two or more respiratory data streams 36.

### Example

In some embodiments, the curves/loops are recorded constantly, and mean values and confidences are computed for a time span (i.e., a day or an hour), and compared to a previous time span, to monitor possibly significant changes during progression.

In some embodiments, the curves/loops are compared on basis of their confidence intervals before and after a change in therapy (e.g., proning, changes in MV settings, or medication).

In some embodiments, the curves/loops are compared on basis of their confidence intervals against other patients (from a data collection), so that the ‘typicalness’ of a patient can be appraised, and other patients which are of significantly close appearance can be retrieved (with therapy and outcome) for reference.

In some embodiments, following a therapeutic change, the resulting curve/loop after the change is compared on basis of their confidence intervals against the induced change expected from the experience with previous similar patients. This is achieved by machine-learning earlier ‘transfer functions’ from applied therapeutic change, together with the transfer confidence interval. The display assists the user in understanding whether a possible deviation from the expected outcome is significant or not.

In some embodiments, disease progression (exacerbation or convalescence) is compared on basis of confidence intervals against the progression expected from the experience with previous similar patients. This is achieved by machine-learning earlier ‘progression functions,’ together with the transfer confidence interval. The display assists the user in understanding whether a possible deviation from the expected progression is significant or not.

In some embodiments, in addition to directly MV-related curves/loops, also other relevant curves/loops from other monitoring devices can be used, in particular from diaphragmatic US with excursion and thickening, capnography, ECG, camera-based life-signs (pulse), etc.

In some embodiments, in addition to confidence from single magnitude variances, also co-variances between magnitudes (e.g., flow and volume, or diaphragmatic excursion and thickening, and so forth) may be considered (i.e., multi-variate confidence intervals). A graphical presentation to the user may be given by displaying covariance-ellipsoids for automatically selected curve regions of significant change.

In some embodiments, the variance itself might provide valuable information, namely about the stability of the patient over a relatively short time. An additional color coding or alike may be used to indicate locations on the curve or loop where the intra-patient variance is much different from what is typically expected.

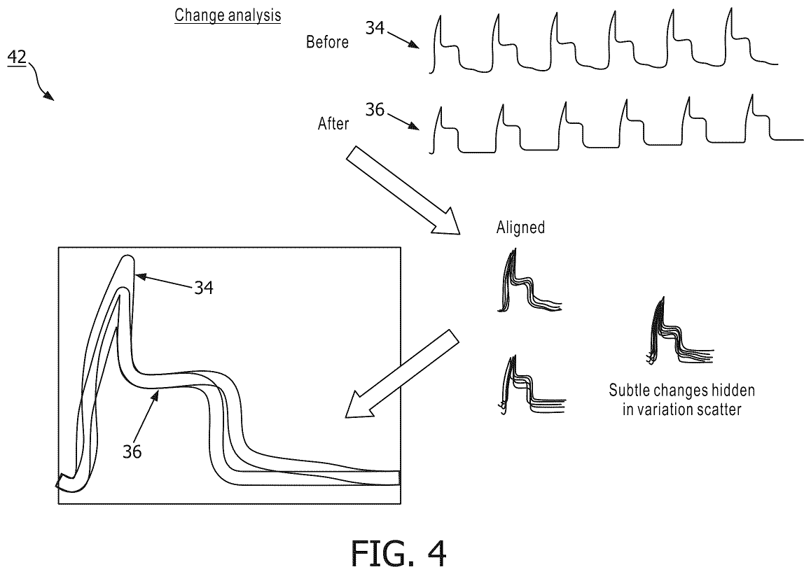

FIG. 4 shows a first example of the common graph 42 showing the two or more respiratory data streams 34, 36. Multiple repeats of the recording of the respiratory cycle of the patient P before and after, such as a therapeutic change (i.e., proning). Alignment by respiratory phase exhibits a fluctuations (i.e., scatter), from which a confidence interval can be derived for each point of the curve. Mean curves before and after are intuitively comparable with confidence encoded as curve thickness. In contrast, direct overlay of all curves may result in clutter hiding subtle changes.

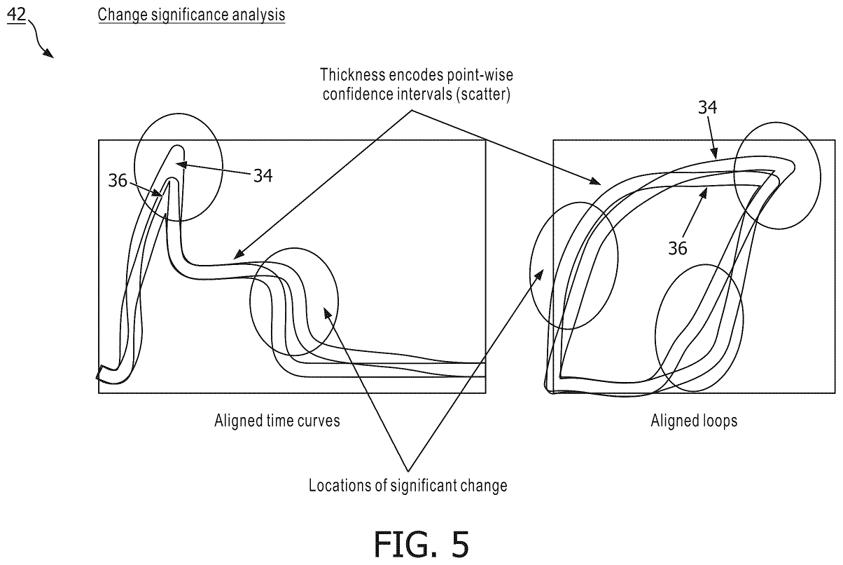

FIG. 5 shows a second example of the common graph 42 showing the two or more respiratory data streams 34, 36. Curves and loops are shown from before and after therapeutic change, with markers showing location of significant changes which are outside of the envelopes of the local confidence intervals.

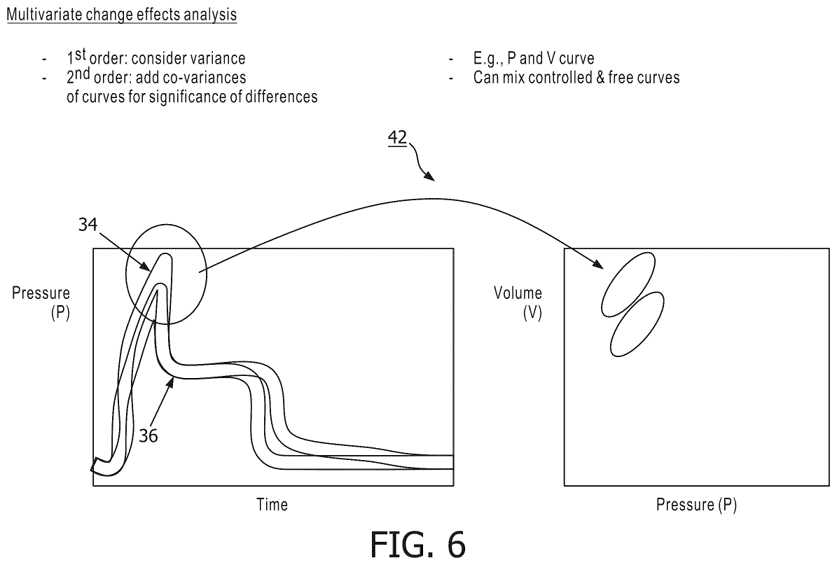

FIG. 6 shows a third example of the common graph 42 showing the two or more respiratory data streams 34, 36. Deviations between two curves/loops may appear insignificant, when the confidence intervals overlap as the confidence estimate is based on the variance of a single magnitude (e.g., flow). The significance may become clear if also co-variances between magnitudes (e.g., flow+volume, or diaphragmatic excursion+thickening) are considered (multi-variate confidence intervals). A graphical presentation to the user may be given by displaying covariance-ellipsoids for automatically selected curve regions of significant change.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A mechanical ventilation device comprising at least one electronic controller configured to:
receive two or more respiratory data streams, each spanning multiple breath cycles;
generate a confidence band over a breath cycle for each data stream based on statistics of the data stream determined from the multiple breaths spanned by the data stream; and
on a display, plot the confidence bands for the data streams on a common graph.

2. The mechanical ventilation device of claim 1, wherein each respiratory data stream comprises a time sequence of measurements of a respiratory parameter, and the generation of the confidence band over the breath cycle for each data stream includes:
partitioning the data stream into segments each corresponding to a breath;
registering the segments in time to produce registered segments;
binning the measurements of the registered segments into time bins of the breath cycle; and
for each time bin of the breath cycle, computing a confidence interval of the binned measurements;
wherein the confidence band comprises a time sequence of the confidence intervals computed for the time bins.

3. The mechanical ventilation device of claim 2, wherein the at least one electronic controller is further configured to:
present a user dialog via which an updated configuration is received for a confidence interval metric used to compute the confidence interval; and
in response to receiving an updated configuration for the confidence interval metric, repeating the computing of the confidence intervals whereby the confidence band is updated in accordance with the updated configuration.

4. The mechanical ventilation device of claim 2, wherein computing the confidence interval of the binned measurements includes:
computing an average or median value of the binned measurements; and
computing a statistical spread of the binned measurements;
wherein the confidence interval comprises the statistical spread around the average or median value.

5. The mechanical ventilation device of claim 4, wherein statistical spread is a standard deviation, a variance, a quartile, a percentage-quantile, or an inter-quartile-range

6. The mechanical ventilation device of claim 1, wherein the received two or more respiratory data streams include:
a first respiratory data stream for a respiratory parameter acquired of a patient receiving mechanical ventilation therapy prior to a modification to the mechanical ventilation therapy, and
a second respiratory data stream for the respiratory parameter acquired of the patient receiving the mechanical ventilation therapy after the modification to the mechanical ventilation therapy.

7. The mechanical ventilation device of claim 6, wherein the modification to the mechanical ventilation therapy comprises proning the patient by moving the patient from a supine position to a prone position.

8. The mechanical ventilation device of claim 6, wherein the at least one electronic controller is further configured to:
determine whether the modification to the mechanical ventilation therapy has produced a statistically significant change in the respiratory parameter acquired of the patient based on detection of a non-overlapping portion of the confidence bands generated for the first and second data streams; and
output an indication of whether the modification to the mechanical ventilation therapy has produced a statistically significant change in the respiratory parameter acquired of the patient based on the automatic determination.

9. The mechanical ventilation device of claim 1, wherein the received two or more respiratory data streams include:
a first respiratory data stream for a respiratory parameter acquired of a patient receiving mechanical ventilation therapy over a first time interval, and
a second respiratory data stream for the respiratory parameter acquired of the patient receiving the mechanical ventilation therapy over a second time interval that is after the first time interval and that is not overlapping the first time interval.

10. The mechanical ventilation device of claim 9, wherein the at least one electronic controller is further configured to:
automatically determine whether there is a change in a condition of the patient based on whether there is a non-overlapping portion of the confidence bands generated for the first and second data streams;
when a change in the curve is automatically determined, at least one of:
output an alarm indicative of the change in the curve; and
output a recommended adjustment to one or more parameters of the mechanical ventilation therapy delivered to the patient.

11. The mechanical ventilation device of claim 1, wherein the received two or more respiratory data streams include:
a first respiratory data stream for a respiratory parameter acquired of a first patient receiving mechanical ventilation therapy, and
a second respiratory data stream for the respiratory parameter acquired of a second patient receiving the mechanical ventilation therapy;
wherein the second patient is different from the first patient.

12. The mechanical ventilation device of claim 1, wherein the received two or more respiratory data streams include:
a first set of two or more respiratory data streams for a corresponding two or more respiratory parameters, and
a second set of two or more respiratory data streams for the corresponding two or more respiratory parameters,
wherein the generation of the confidence band over the breath cycle for each data stream based on statistics of the data stream determined from the multiple breaths spanned by the data stream includes:
for the first set of two or more respiratory data streams, determining the confidence band based on a covariance between the two or more respiratory data streams of the first set of two or more respiratory data streams; and
for the second set of two or more respiratory data streams, determining the confidence band based on the covariance between the two or more respiratory data streams of the second set of two or more respiratory data streams.

13. A mechanical ventilation monitoring or assessment method, comprising, with at least one electronic controller:
receiving two or more respiratory data streams, each spanning multiple breath cycles;
generating a confidence band over a breath cycle for each data stream based on statistics of the data stream determined from the multiple breaths spanned by the data stream; and
on a display, plotting the confidence bands for the data streams on a common graph.

14. The mechanical ventilation monitoring or assessment method of claim 13, wherein the received two or more respiratory data streams include:
a first respiratory data stream for a respiratory parameter acquired of a patient receiving mechanical ventilation therapy prior to a modification to the mechanical ventilation therapy, and
a second respiratory data stream for the respiratory parameter acquired of the patient receiving the mechanical ventilation therapy after the modification to the mechanical ventilation therapy.

15. The mechanical ventilation monitoring or assessment method of claim 14, wherein the modification to the mechanical ventilation therapy comprises proning the patient by moving the patient from a supine position to a prone position.
